(19) United States
(12) Patent Application Publication
(10) Pub. No.: US 2016/0000456 A1
(43) Pub. Date: Jan. 7, 2016

(54) Method and Apparatus for Use in Removing a Blade From a Scalpel

(71) Applicant: QLICKSMART PTY LTD, Brisbane, QLD (AU)

(72) Inventor: Kim Quek, Brisbane (AU)

(21) Appl. No.: 14/767,695

(22) PCT Filed: Feb. 18, 2014
(86) PCT No.: PCT/AU2014/000135

§ 371 Date: Aug. 13, 2015

(30) Foreign Application Priority Data
Feb. 20, 2013 (AU) 2013900568

(51) Int. Cl. A61B 17/3217

(52) U.S. Cl. CPC A61B 17/3217

(57) ABSTRACT

An apparatus is provided for safely removing a blade from a scalpel using a scalpel blade remover. The apparatus includes a combination of a housing having a formation defining a socket of predetermined depth, and at least one elongate scalpel blade remover. The socket, which may be a tubular portion of circular cross section, is dimensioned to receive therein a lower portion of the blade remover such that the blade remover is retained in an upright or inclined position in the socket when the housing is in an operative orientation. The blade remover can be orientated within the socket at a variable azimuth. The housing may be a case including two hinged parts, with the socket in one of the hinged parts, and a sharps holder in the other hinged part.

### TECHNICAL FIELD

The present invention relates to a method and apparatus for use in removing a blade from a scalpel.

In a particular embodiment, the invention relates to a combination of such apparatus with a needle counter.

### BACKGROUND ART

Reference to any prior art in this specification is not, and should not be taken as, an acknowledgement or suggestion that the prior art forms part of the common general knowledge in the art in any particular country.

International patent application no. WO 2004/093706 discloses a single-use disposal scalpel blade remover [hereafter referred to simply as a ‘blade remover’]. That blade remover is of elongate form, and has a slot adapted to receive the blade of a scalpel. In use, the tang of a scalpel having a blade mounted thereon is inserted into the slot. The insertion of the tang actuates a cam-operated mechanism which separates the heel of the blade from the tang as the tang is pushed into the device. When the tang has been inserted to a predetermined extent, a detent or pawl prevents retraction of the blade. The scalpel is then withdrawn from the device, causing the blade to be stripped from the tang. The blade is safely retained within the device, which can then be disposed of. The disclosure of application WO 2004/093706 is incorporated herein by reference.

The blade remover of WO 2004/093706 can be used single-handedly. However, it is necessary for the blade remover to be held securely when inserting the scalpel tang and blade into the device. Users may be tempted to hold the blade remover in one hand, and insert the scalpel/blade with the other hand. This is a dangerous practice which could result in a sharps injury, as the blade at the leading edge of the scalpel may pierce the hand holding the blade remover if the blade misses the slot at the initial insertion step.

During surgical operations, it is crucially important to keep count of the number of suture needles used, to avoid needles being inadvertently left in the patient. Needle counters are normally used for this purpose. A needle counter is typically in the form of a foam or penetrable mat having numbered portions thereon. After each suture needle is used in the operation, it is pinned to the mat in a respective numbered portion, sequentially. In this manner, the surgeon or theatre nurse can tell at a glance how many used suture needles have been recovered. An example of a needle counter mat can be found in U.S. Pat. No. 4,418,821, the disclosure of which is incorporated herein by reference.

Such needle counters are normally provided as separate items, adding to the cost and complexity of surgical operations.

Some needle counters incorporate a scalpel blade removing mechanism or device but are considered inherently unsafe as they do not provide a firm and secure support for the blade removing mechanism or device in use. In particular, such needle counter/blade remover combinations are not able to resist the horizontal pushing and twisting forces applied to the device in use. Consequently, the operator may have to hold the combination with one hand while manoeuvring the bladed scalpel with the other hand, increasing the likelihood of a sharps injury.

### SUMMARY OF INVENTION

It is an aim of the invention to overcome or ameliorate one or more of the disadvantages mentioned above, or at least provide the consumer with a useful choice.

With the foregoing in view, the present invention in one form, provides an apparatus for use in removing a blade from a scalpel, the apparatus comprising a housing having a formation defining a socket with a circular cross-section of predetermined depth, the socket being dimensioned to receive therein a lower portion of an elongate scalpel blade remover such that the blade remover is retained in an upright or inclined position in the socket when the housing is in an operative orientation, and whereby the blade remover can be orientated within the socket at a variable azimuth.

In another form, the invention provides an apparatus for use in removing a blade from a scalpel, the apparatus comprising a combination of a housing having a formation defining a socket of predetermined depth and at least one elongate scalpel blade remover, wherein the socket is dimensioned to receive therein a lower portion of the blade remover such that the blade remover is retained in an upright or inclined position in the socket when the housing is in an operative orientation, and wherein the blade remover can be orientated within the socket at a variable azimuth.

Typically, the housing is a case having two hinged parts.

An adhesive patch is preferably provided on an exterior portion of at least one of the parts of the case to secure it to a surface in a non-slip manner.

In the preferred embodiment, the formation defining the socket is located in one of the hinged parts, and a sharps holder is located in the other hinged part.

The sharps holder may have a suture needle counter. The suture needle counter may be in the form of a sponge or sponge-like mat having numbered portions thereon, or other suitable markings, for receiving respective used suture needles or sharps.

Advantageously, the case is dimensioned to hold multiple blade removers therein.

Preferably, the formation defining the socket is formed integrally with the housing. This formation may be a tubular member extending from a base on the respective hinged part, and having a length equal to the predetermined depth. Alternatively, the formation may be a cylindrical recess of the predetermined depth in the respective hinged part of the case.

The elongate device for removing the blade from the scalpel is preferably the blade remover described in international patent application no. WO 2004/093706 and sold by Qlicksmart Pty Ltd. This blade remover comprises
a blade remover housing;
an actuator having a proximal end, the actuator being operatively juxtaposed with the blade remover housing and moveable relative to the blade remover housing from a proximal position, whereat an opening is formed between the actuator and the blade remover housing for receiving therein a blade on a tang of a scalpel, to a distal position;
a blade deflecting mechanism for automatically separating a heel of the blade from the tang of the scalpel as the actuator is moved to the distal position; and
a retention mechanism for engaging the actuator and retaining it in the distal position;
wherein in use, when the blade on the tang is inserted in the opening and the actuator is pushed forward by the tang to its distal position,
the blade deflecting mechanism automatically separates the heel of the blade from the tang of the scalpel,
the retention mechanism engages the actuator and retains it in its distal position and limits rearward movement of the blade, and
   upon withdrawal of the tang from the scalpel blade remover, the blade is stripped from the tang and retained between the actuator and the housing.
   However, other suitable blade removers may be used.

The apparatus of the present invention serves as a safe and secure mount for the blade remover during the process of insertion of the blade into the blade remover.

In yet another form, the invention provides a method of removing a blade from a scalpel using the apparatus of the type described above, comprising the steps of
placing the housing on a surface so that the socket faces upwardly,
inserting a lower portion of the blade remover into the socket,
orientating the blade remover in the socket to a desired azimuth,
permitting the blade remover to rest against a side of the socket such that the blade remover is inclined to the horizontal,
inserting a tang of a scalpel having a blade mounted thereon into the blade remover, pivoting the blade remover to a generally upright orientation, and pressing the scalpel further downwardly into the blade remover such that a blade removing mechanism in the blade remover engages the blade, and
removing the scalpel from the blade remover while the blade is retained in the elongate device.

The apparatus of the present invention has several advantages, including:
The socket provides a handy, safe and secure formation in which to locate the blade remover during use, thereby reducing the risk of a sharps injury.
The circular section of the socket in which the blade remover is inserted allows the blade remover to be orientated at a variable azimuth. Consequently, the blade remover can be orientated to suit both left-handed and right-handed persons. The blade remover can also be orientated to suit the particular position in which the operator is standing.
The socket enables the blade remover to be used single-handedly, while complying with workplace health and safety regulations and practices.
The predetermined depth of the socket determines the angle at which the blade remover is orientated when it is placed in the socket and permitted to rest against the socket wall. The depth can be chosen so that the blade remover is orientated at the desired inclination.
The circular socket not only permits the blade remover to be inclined by resting against a socket wall, but permits the blade remover to be pivoted to a vertical orientation to facilitate the step of pushing the scalpel into the blade remover. The scalpel can be pressed downwardly into the blade remover with only one hand, thereby freeing the other hand from having to hold the apparatus.
Combining the apparatus with a needle counter reduces the amount of space required on the tray or table top used in the operation.
The combination of the apparatus and the needle counter reduces the overall costs of manufacturing, storing and supplying the two respective products.

Any of the features described herein can be combined in any combination with any one or more of the other features described herein within the scope of the invention.

### BRIEF DESCRIPTION OF DRAWINGS

One or more embodiments of the invention will now be described, by way of example, with reference to the following drawings, in which:

FIG. 1 is a perspective view of the apparatus of the present invention, in a closed inverted configuration.

FIG. 2 is a perspective view of the apparatus of the present invention in an open configuration.

FIG. 3 is a perspective view of the apparatus of FIG. 2 in use.

FIGS. 4 to 7 are perspective views showing the apparatus of FIG. 2 being used in a scalpel blade removal process.

FIG. 8 is a perspective view showing removal of a scalpel from a blade remover.

FIG. 9 is a perspective view illustrating a removed blade captured in the blade remover.

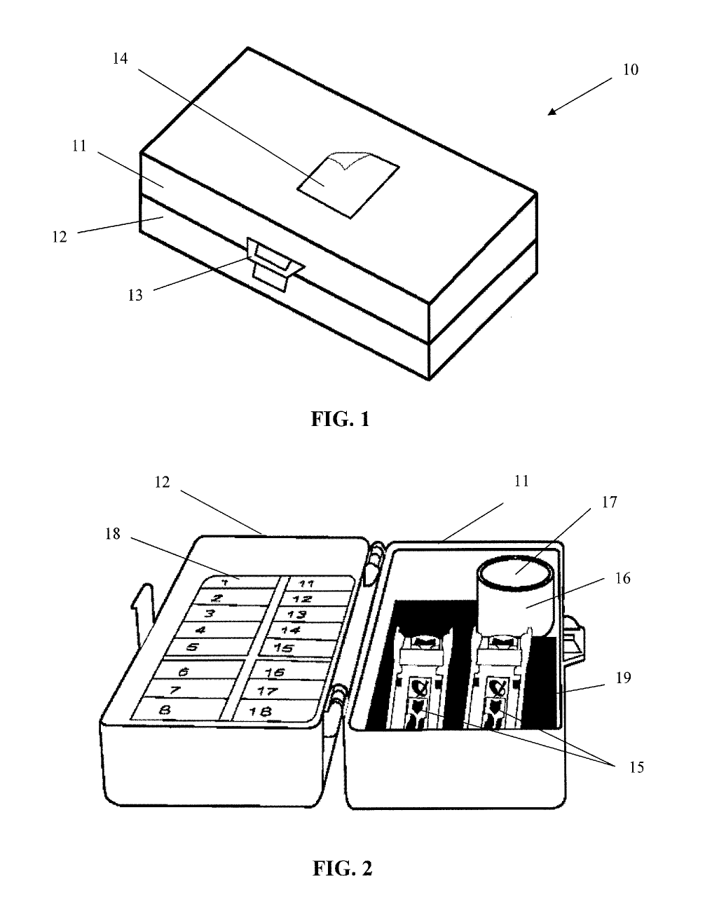

Preferred features, embodiments and variations of the invention may be discerned from the following detailed description which provides sufficient information for those skilled in the art to perform the invention. The detailed description is not to be regarded as limiting the scope of the invention in any way.

### DESCRIPTION OF EMBODIMENT(S) OF THE INVENTION

As shown in the drawings, in one embodiment, the invention is in the form of a case 10 comprising two hinged portions 11, 12 releasably secured by a clasp 13. Typically, the case 10 is made of moulded plastics material, and each hinged portion 11, 12 is generally in the form of a shallow rectangular tray.

A length of double sided tape 14 may be provided on the outside of the portion 11, and also on the outside of the portion 12 (not shown). Before the case 10 is placed in position, the top layer of each length of tape is peeled off to expose the adhesive surface. When the case 10 is opened and placed on a drape, tray or table top at the desired location, the adhesive tape 14 assists in retaining the case firmly, but releasably, in position.

The portion 11 may suitably contain a desired number of blade removers 15. These blade removers are preferably of the type described in international patent application no. WO 2004/093706. As described in WO 2004/093706, each such blade remover comprises
a blade remover housing;
an actuator having a proximal end, the actuator being operatively juxtaposed with the blade remover housing and moveable relative to the blade remover housing from a proximal position, whereat an opening is formed between the actuator and the blade remover housing for receiving therein a blade on a tang of a scalpel, to a distal position;
a blade deflecting mechanism for automatically separating a heel of the blade from the tang of the scalpel as the actuator is moved to the distal position; and
a retention mechanism for engaging the actuator and retaining it in the distal position;
wherein in use, when the blade on the tang is inserted in the opening and the actuator is pushed forward by the tang to its distal position,
the blade deflecting mechanism automatically separates the heel of the blade from the tang of the scalpel,
the retention mechanism engages the actuator and retains it in its distal position and limits rearward movement of the blade, and
upon withdrawal of the tang from the scalpel blade remover, the blade is stripped from the tang and retained between the actuator and the housing

In the illustrated embodiment, two blade removers 15 are provided in the tray, each blade remover being adapted to remove one blade from a scalpel.

The instructions for use of the case 10 may optionally be printed and illustrated in the base of portion 11 to assist the user.

Hinged portion 11 also contains a tubular formation 16 which preferably is integrally formed with the portion 11. The tubular portion 16 provides a socket 17 of generally circular cross section. Although the socket 17 in the illustrated embodiment is provided by the short tubular portion 16, other formations may be used to provide the circular socket, such as a cylindrical recess in a thick solid portion, or a closed tubular member depending downwardly from an aperture in an upper plate on the portion 11.

The other portion 12 of the case 10 comprises a needle counter mat in the form of a foam, sponge or other penetrable layer having numbered portions 18 thereon. Such needle counter mats are known in the art and need not be described in detail in this application.

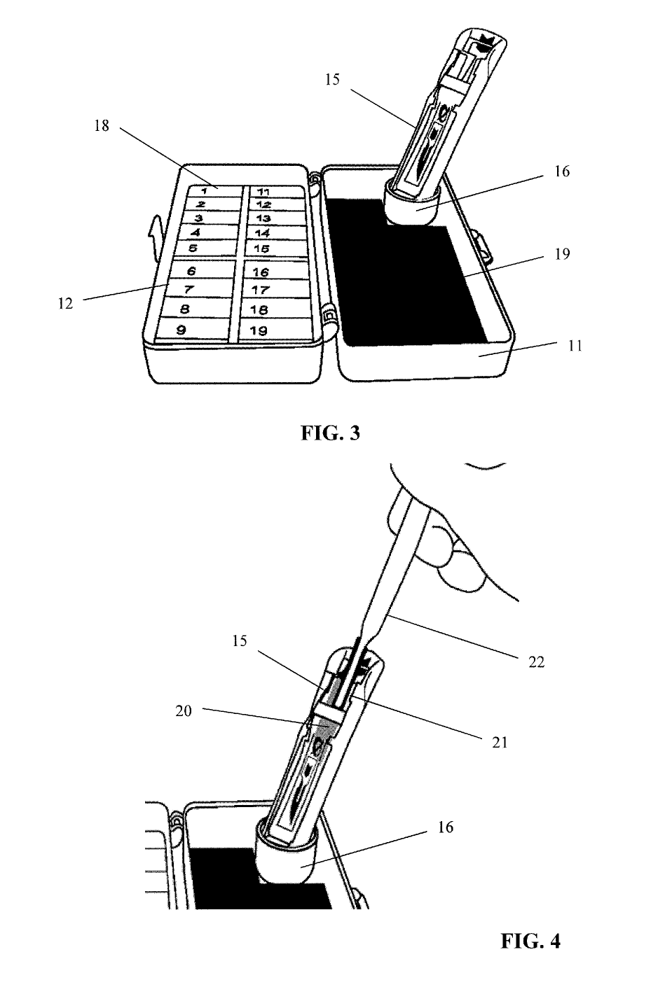

FIGS. 3 to 7 illustrate the use of the case 10 in the safe removal of a blade from a scalpel. First, a blade remover 15 is inserted in the socket 17, and allowed to rest against the wall of the socket so that it is orientated at an oblique angle. Due to the circular nature of the socket 17, the blade remover can be orientated at an infinitely variable azimuth, i.e. the horizontal angle. Thus, the azimuth angle of the blade remover 15 can be adjusted to suit left-handed and right-handed persons, and the position of the user.

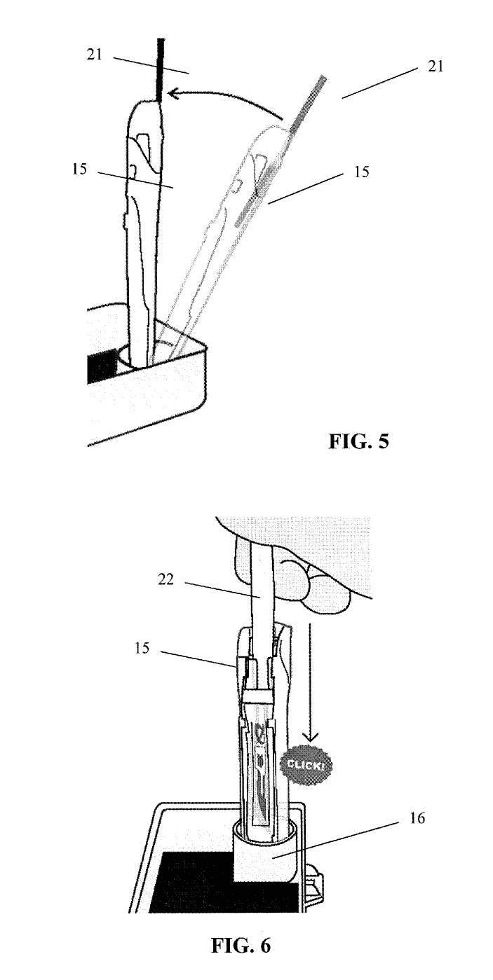

As shown in FIG. 4, a blade 20 is mounted on the tang 21 of a scalpel 22. The blade and tang are inserted into the appropriate slot in the blade remover 15. Using the scalpel as a lever, the blade remover 15 is then tilted to a generally upright or vertical position as shown in FIG. 5.

With blade remover 15 in a generally upright orientation, the scalpel 22 is then pressed downwardly until a click is heard. This click sound signifies that a detent or pawl in the blade remover 15 has engaged the trailing edge of the blade 20, preventing the blade from being retracted from the blade remover 15.

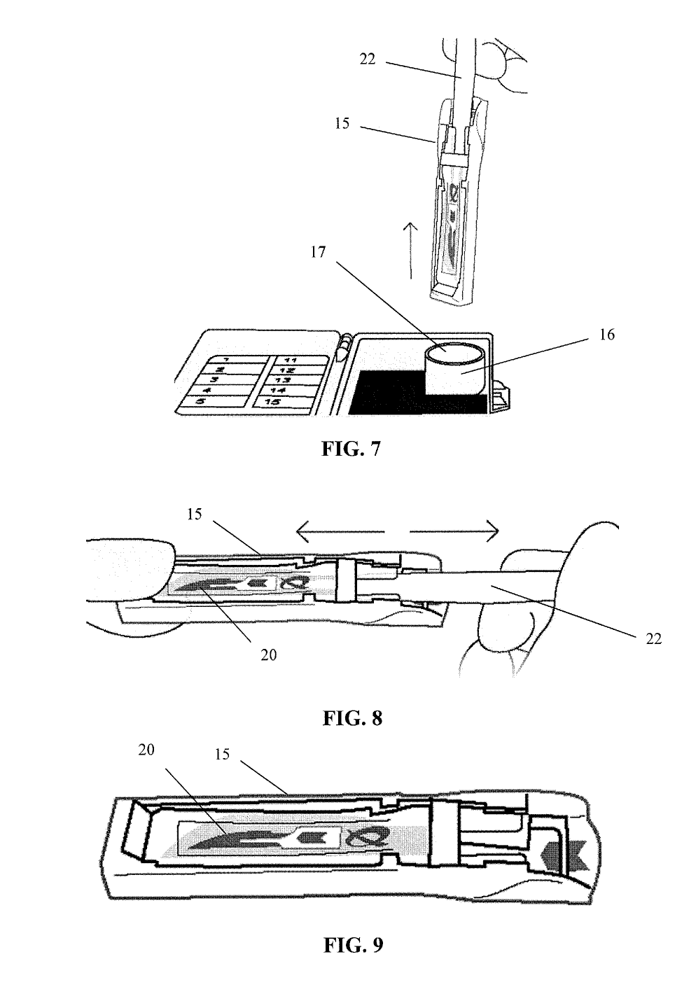

The blade remover 15 is made of lightweight plastics material and, as the blade 20 is now captured within the blade remover 15, the blade remover is effectively connected to the scalpel 22. Using the scalpel 22, the user may now lift the blade remover 15 out of the socket, as shown in FIG. 7.

It is to be noted that the positioning of the blade remover, insertion of the blade into the blade remover so that the blade is captured therein, and the removal of the blade remover from the socket are all performed singlehandedly. This means that the unused hand can be positioned at a safe distance away, thereby eliminating the risk of sharps injury during this process.

After the blade remover 15 (with the scalpel still mounted therein) has been removed from the socket 17, the blade 20 is safely retained within the blade remover. The blade remover can then be grasped with the other hand, and the scalpel 22 retracted from the blade remover. This strips the blade 20 from the tang 21 of the scalpel, leaving the blade 20 within the blade remover 15 as shown in FIG. 9. The blade remover and the removed blade can then be disposed of.

It is to be understood that the invention is not limited to specific features shown or described since the means herein described are preferred forms of putting the invention into effect. The invention includes modifications as would be apparent to those skilled in the art. For example, the socket 17 may be of a cross-section other than circular, e.g. elliptical or polygonal.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

### INDUSTRIAL APPLICATION

The invention provides an improved method and apparatus for use in removing a blade from a scalpel, as well as a combination of such apparatus with a needle counter, which can be used in a safe manner in surgeries, laboratories and similar applications.

1. An apparatus for use in removing a blade from a scalpel, the apparatus comprising a housing having a formation defining a socket with a circular cross-section of predetermined depth, the socket being dimensioned to receive therein a lower portion of an elongate scalpel blade remover such that the elongate scalpel blade remover is retained in an upright or inclined position in the socket when the housing is in an operative orientation, and whereby the elongate scalpel blade remover can be orientated within the socket at a variable azimuth.

2. An apparatus for use in removing a blade from a scalpel, the apparatus comprising a combination of (i) a housing having a formation defining a socket of predetermined depth and (ii) at least one elongate scalpel blade remover, wherein the socket is dimensioned to receive therein a lower portion of the at least one elongate scalpel blade remover such that the at least one elongate blade remover is retained in an upright or inclined position in the socket when the housing is in an operative orientation, and wherein the at least one elongate scalpel blade remover can be orientated within the socket at a variable azimuth.

3. The apparatus as claimed in claim 1, wherein the housing is a case comprising two hinged parts.

4. The apparatus as claimed in claim 3, further comprising an adhesive patch on an exterior portion of at least one of the parts of the case to secure it to a surface.

5. The apparatus as claimed in claim, wherein the formation defining the socket is located in one of the hinged parts, and a sharps holder is located in the other hinged part.

6. The apparatus as claimed in claim 5, wherein the sharps holder has a suture needle counter.

7. The apparatus as claimed in claim 3 wherein the case is dimensioned to hold multiple blade removers therein.

8. The apparatus as claimed in claim 3, wherein the formation defining the socket is formed integrally with the housing.

9. The apparatus as claimed in claim 3, wherein the formation defining the socket is a tubular portion.

10. The apparatus as claimed in claim 2, wherein the formation defining the socket is a tubular portion with a circular cross-section.

11. The apparatus as claimed in claim 1, wherein the at least one elongate scalpel blade remover comprises
a blade remover housing;
an actuator having a proximal end, the actuator being operatively juxtaposed with the at least one elongate scalpel blade remover housing and moveable relative to the blade remover housing from a proximal position, whereat an opening is formed between the actuator and the blade remover housing for receiving therein a blade on a tang of a scalpel, to a distal position;
a blade deflecting mechanism for automatically separating a heel of the blade from the tang of the scalpel as the actuator is moved to the distal position; and
a retention mechanism for engaging the actuator and retaining it in the distal position;
wherein in use, when the blade on the tang is inserted in the opening and the actuator is pushed forward by the tang to its distal position,
the blade deflecting mechanism automatically separates the heel of the blade from the tang of the scalpel,
the retention mechanism engages the actuator and retains it in its distal position and limits rearward movement of the blade, and
upon withdrawal of the tang from the at least one scalpel blade remover, the blade is stripped from the tang and retained between the actuator and the housing.

12. A method of removing a blade from a scalpel using the apparatus as claimed in any preceding claim, comprising the steps of
placing the housing on a surface so that the socket faces upwardly,
inserting a lower portion of the blade remover into the socket,
orientating the blade remover in the socket to a desired azimuth,
permitting the blade remover to rest against a side of the socket such that the blade remover is inclined to the horizontal,
inserting a tang of a scalpel having a blade mounted thereon into the blade remover, pivoting the blade remover to a generally upright orientation, and pressing the scalpel further downwardly into the blade remover such that a blade removing mechanism in the blade remover engages the blade, and
removing the scalpel from the blade remover while the blade is retained in the elongate device.

13. The method as claimed in claim 11, further comprising adhering the housing to the surface in a non-slip manner before inserting the lower portion of the blade remover into the socket.
